(19) United States
(12) Patent Application Publication
(10) Pub. No.: US 2023/0013131 A1
(43) Pub. Date: Jan. 19, 2023

(54) DYNAMIC SHOULDER BRACE

(71) Applicant: ADONIS MEDICAL TECHNOLOGIES LTD., Edmonton (CA)

(72) Inventors: Ming CHAN, Edmonton (CA); Taylor MURPHY, Edmonton (CA); Dhruvi DAVE, Edmonton (CA); Jim TOLLER, Edmonton (CA); Mark Vernon FEDORAK, Edmonton (CA); Liam Joseph ABRAMS, Edmonton (CA)

(21) Appl. No.: 17/378,514

(22) Filed: Jul. 16, 2021

(51) Int. Cl. A61F 5/37; A61F 5/01; A61H 1/02

(52) U.S. Cl. CPC A61F 5/3753; A61F 5/013; A61F 5/3761; A61H 1/0277; A61H 1/0281; A61F 2005/0158; A61F 2005/0155; A61H 2201/1238

(57) ABSTRACT

A novel shoulder brace that can provide active dynamic support to an injured shoulder while providing the capability to also raise the attached arm and support the arm in an elevated position. The brace can be used both in conjunction with a rehabilitation intervention program to maintain or restore range of motion and strength and to assist in functional tasks at work and at home. The brace works by supporting a locking or ratchet mechanism connected to an arm cuff from a chest piece that, when desired, the user can move the arm connected to an injured shoulder and supported by the arm cuff from a normal side resting position to an elevated position 90 degrees from the resting position while transferring the load created by the arm and whatever the hand is holding back to the chest piece and to the torso of the user. A release mechanism can be used to release the locking or ratchet mechanism to let the user lower their arm back to a resting position.

### TECHNICAL FIELD

The present disclosure is related to the field of shoulder orthosis and, in particular, restoring function and motion to an injured shoulder to assist with rehabilitation and restoration of the ability to complete daily living and work-related functions without additional assistance.

### BACKGROUND

Traumatic nerve injuries affecting the arm and shoulder are common, occurring in up to 3% of trauma patients in North America. Many of those patients are at their prime and active in the work force prior to injury. Some of those injuries result in weakness or paralysis of the shoulder joint that have a drastic impact on activities of daily living and the patient's ability to return to work. Apart from major functional limitations, the immobile shoulder can also be very painful. Because of the ball and socket structure of the shoulder joint, it is prone to developing stiffness and can easily become “frozen”. Therefore, maintaining or restoring the range of motion expeditiously after nerve injury is a high priority. Although surgery has been successful in restoring function of the elbow, wrist and hand, surgical restoration of shoulder function has proven to be more challenging. Major reasons for this are the needs to overcome substantial weight of the arm and intrinsic instability of the shoulder joint when the periscapular muscles are weakened. Furthermore, the long lever arm places the shoulder at a mechanical disadvantage compared to the distal joints.

Injury to nerves in the arm through accidents such as falls and motor vehicle accidents are very common. Those afflicted are often young men who are otherwise healthy, productive members in society (Noble, Munro et al. 1998, Dubuisson and Kline 2002). Among these conditions, brachial plexus injury is particularly devastating because nerve disruptions occurring proximally in the shoulder and neck can have a major impact on the entire arm. Therefore, not surprisingly, a large proportion of brachial plexus injury patients are unable to return to work, have to be assigned to modified duties or undergo retraining to accommodate the limitations imposed by the injury (Kandenwein, Kretschmer et al. 2005, Kretschmer, Ihie et al. 2009, Franzblau, Shauver et al. 2014).

Of the different parts of the brachial plexus, injury to the upper trunk of the brachial plexus is among the most common (Dubuisson and Kline 2002). Upper trunk brachial plexus injury particularly affects shoulder and elbow function. While it is fortunate that their wrist and hand function remain intact, deficits at the shoulder and elbow limit the patient's ability to reach or and place the hand at the target workspace. As reaching and holding constitute a fundamental requirement in most arm activities, it is not surprising that many individuals with brachial plexus injury engaged in physically demanding jobs are unable to return to work (Kretschmer, Ihie et al. 2009).

The versatility of the shoulder is in a large part attributable to its capability of multidirectional movements. To achieve the full range of movements, three joints (glenohumeral, acromioclavicular and stenoclavicular) and one articulation (scapulothoracic) in the shoulder girdle need to move in unison and with fluid complementarity in multiple planes. To achieve optimal function, the shoulder also requires strong tendino-muscular support as the joints are intrinsically unstable (Merolla, Cerciello et al. 2015). In addition to the fact that the glenoid fossa is too shallow a socket to provide good bony support to the arm, the situation is further exacerbated when the patient attempts to carry a heavy load with an outstretched arm.

With those challenges in mind, one can appreciate why functional limitations may quickly ensue following injuries to nerve, muscle, and associated soft tissue in the shoulder region. Indeed, even with brief immobility, the shoulder joints and scapulothoracic articulation can stiffen up easily, resulting in a “frozen shoulder”. Paralysis of the muscle further exacerbates the situation when the strength needed to keep the shoulder moving is limited or absent. Muscle weakness may also contribute to shoulder instability. Therefore, in order to produce meaningful therapeutic benefits to patients with shoulder injury, all 3 elements—maintaining joint mobility, ensuring adequate stability, and support of the shoulder through a wide range of movement—have to be taken into account.

The brachial plexus consists of 5 spinal roots (C5 to T1), 3 trunks (upper, middle and lower), 2 divisions, and 3 cords (lateral, posterior and medial) before giving rise to peripheral branches of nerves. The upper trunk, among the most commonly injured part of the brachial plexus, receives contributions from the C5 and C6 spinal roots that play a particularly important role in many shoulder girdle muscles. Major examples include the deltoid and supraspinatus for shoulder flexion and abduction and infraspinatus for shoulder external rotation (Limthongthang, Bachoura et al. 2013).

Although the rhomboids help to stabilize the scapula, weakness in that muscle can be compensated by the trapezius. The trapezius is a large, powerful muscle that is crucial in stabilizing and guiding the scapula during shoulder retraction. Medial winging of the scapula is usually not an issue in upper trunk brachial plexus injury because the inferior portion of serratus anterior that keeps the scapula well apposed to the thoracic cage is primarily innervated by the C7 root (Bertelli and Ghizoni 2005). Therefore, the most debilitating consequence of upper trunk brachial plexus injury is the loss of shoulder abduction and external rotation. Furthermore, as a result of marked wasting of the deltoid, subluxation of the glenohumeral joint causing instability and pain frequently occurs.

Although upper trunk brachial plexus injury can also affect elbow flexion, functionality of the elbow is relatively simple. Furthermore, a number of good surgical and non-surgical options already exist. Therefore, restoration of elbow flexion is a less daunting task (Oberlin, Ameur et al. 2002, Fox and Mackinnon 2011).

Despite the functional importance of the shoulder joint, there are surprisingly few shoulder support or bracing systems available. By far, the most commonly used is shoulder swath or sling that wraps around the arm. While it helps to reduce subluxation of the glenohumeral joint, a major downside is that it also constrains shoulder moments and could hasten the development of frozen shoulder. There are also shoulder stabilizers made of nylon and cloth materials that strap around the shoulder. Although these allow the patient to use the forearm and hand unimpeded, they also limit shoulder movements.

To fill the void of providing dynamic support to the shoulder and to power movements, attempts have been made to design motorized exoskeletons either mounted on a wearable brace on the patient or attached to a stationary robotic setup (Sicuri, Porcellini et al. 2014). Apart from being immobile and expensive, perhaps the most severe limitation of those devices is that they are cumbersome and cannot be easily incorporated into daily routines. Therefore, not surprisingly, they are rarely used.

Although a few braces are available on the market, none is capable of dynamically supporting the shoulder for activities of daily living and work-related functional demands. The dynamic shoulder brace described herein is primarily geared towards patients with injury involving the upper trunk portion of the brachial plexus. Although these patients have full use of their wrist and hand, they are unable to raise their arm at the shoulder. Even with successful surgery and nerve repair, it often takes years for patients to recover functional movements at the shoulder.

It is, therefore, desirable to provide a method, system, and apparatus for providing arm support using a shoulder brace that overcomes the shortcomings of the prior art.

### SUMMARY

To address the limitations in the currently available options, a fundamentally different approach to designing a brace was taken that would meet the crucial requirements of providing stability to the shoulder, allow the patient to mobilize the joint early after injury, and provide support to the arm in a wide range of positions. Also, through in-depth consultation with patients and clinicians, it was determined that for the brace to be widely acceptable it needs to be light, easy to put on and take off, and relatively inexpensive. Because brachial plexus injury is a unilateral condition in the vast majority of cases, the patient can use the opposite hand to operate and adjust the brace settings, including operating the pull ring to release the locking lever. Apart from functional tasks, the dynamic shoulder brace can also be used early after nerve injury for range of motion exercises to minimize the possibility of developing frozen shoulder.

To allow the patient to mobilize the shoulder, the ratchet hinge joint can be locked at regular steps up to 90 degrees of abduction. A ratchet hinge joint bracket, mounted below the axilla can be housed in a low friction vertical support member, to allow a second axis of movement. It can enable the patient to position the arm to the front, side and back, thereby covering the entire workspace. In addition to providing support to the upper arm, when the shoulder is elevated, the brace also allows the elbow to work in a gravity eliminated position. The dynamic shoulder brace can be designed to be light-weight such that it can be worn under a coat or jacket. The chest piece and upper arm cuff can be made of polypropylene that can be perforated for ventilation. Similarly, the 2-axis ratchet hinge joint, which can be made of 316 stainless steel and nylon or PTFE, can also be designed for orthotic use.

Broadly stated, in some embodiments, a shoulder brace can be provided for a person having a torso, a shoulder and an arm attached to the shoulder, the shoulder brace comprising: a chest piece configured for fitment and attachment to the torso; an arm cuff configured for fitment and attachment to the arm; an arm lifting mechanism operatively coupling the arm cuff to the chest piece, the arm lifting mechanism configured for upward movement of the arm cuff from a lowered position to an elevated position and holding the arm cuff in the elevated position thereby maintaining the arm at the elevated position; and a release mechanism configured for acting on the arm lifting mechanism to enable downward movement of the arm cuff from the elevated position to enable the lowering of the arm.

Broadly stated, in some embodiments, the chest piece can further comprise a shoulder support that supports the chest piece from the shoulder.

Broadly stated, in some embodiments, the chest piece can comprise one or more tightening straps configured for releasable attachment of the chest piece to the torso.

Broadly stated, in some embodiments, the arm cuff can comprise a cuff strap configured for releasable attachment of the arm cuff to the arm.

Broadly stated, in some embodiments, the arm lifting mechanism can comprises: a vertical support member operatively coupled to the chest piece; a vertical sliding shaft slidably disposed within the vertical support member; a shaft attachment slidably operatively coupled to the arm cuff; an arm support shaft slidably disposed within the shaft attachment slide; and a ratchet mechanism operatively coupling the vertical sliding shaft to the arm support shaft, the ratchet mechanism configured to enable upward rotational movement of the arm support shaft relative to the vertical sliding shaft when the arm cuff is moved from the lowered position to the elevated position.

Broadly stated, in some embodiments, the ratchet mechanism can comprise: a ratchet wheel operatively coupled to one of the vertical sliding shaft and the arm support shaft; and a ratchet housing operatively coupled to the other of the vertical sliding shaft and the arm support shaft, the ratchet housing comprising a ratchet pawl, the ratchet pawl comprising a biasing element configured to urge the ratchet pawl to engage the ratchet wheel.

Broadly stated, in some embodiments, the release mechanism can comprise: a lever operatively coupled to the ratchet pawl; and a pull string operatively coupled to the lever, the pull string configured to rotate the lever, wherein pulling the pull string disengages the ratchet pawl from the ratchet wheel enabling downward rotational movement of the arm support shaft relative to the vertical sliding shaft, and thereby enabling the arm cuff to move downward from the elevated position.

Broadly stated, in some embodiments, wherein the pull string can be disposed on the chest piece.

Broadly stated, in some embodiments, the release mechanism can comprise: a lever operatively coupled to the ratchet pawl; and an actuator operatively coupled to the lever, the actuator configured to rotate the lever, wherein activation of the actuator disengages the ratchet pawl from the ratchet wheel enabling downward rotational movement of the arm support shaft relative to the vertical sliding shaft, and thereby enabling the arm cuff to move downward from the elevated position.

Broadly stated, in some embodiments, wherein the actuator can comprise an electrically-controlled solenoid.

Broadly stated, in some embodiments, the shoulder brace can further comprise a battery and a wired remote button operatively coupled to the solenoid wherein depressing the wired remote button can activate the actuator to release the ratchet pawl from the ratchet wheel.

Broadly stated, in some embodiments, the shoulder brace can further comprise a wirelessly-controlled brace controller disposed thereon, the brace controller operatively coupled to the solenoid of the actuator, the brace controller configured to activate the actuator upon receiving a wireless signal.

Broadly stated, in some embodiments, the shoulder brace can further comprise one or both of a wireless remote and a smart phone, each of the wireless remote and the smart phone configured to transmit the wireless signal to the brace controller.

Broadly stated, in some embodiments, the arm lifting mechanism can comprise: a vertical support member operatively coupled to the chest piece; a vertical sliding shaft slidably disposed within the vertical support member; a shaft attachment slidably operatively coupled to the arm cuff; an arm support shaft slidably disposed within the shaft attachment slide, the arm support shaft rotatably coupled to the vertical sliding shaft; and a hydraulic system operatively coupling the vertical sliding shaft to the arm support shaft, the hydraulic system configured to enable upward rotational movement of the arm support shaft relative to the vertical sliding shaft thereby enabling movement of the arm cuff from the lowered position to the elevated position.

Broadly stated, in some embodiments, the hydraulic system can comprise: a piston slidably disposed in a housing, the piston operatively coupled to the arm cuff; a first fluid path comprising a check valve configured to permit flow of the hydraulic fluid therethrough to the cylinder when the piston is drawn upward in the cylinder via the raising of the arm cuff to the elevated position, the check valve further configured to prevent the hydraulic fluid from flowing back therethrough from the cylinder; and a second fluid path comprising a release valve configured to permit flow of the hydraulic fluid from the cylinder when the release valve is opened, wherein the piston moves downward in the cylinder thereby enabling the arm cuff to lower from the elevated position.

Broadly stated, in some embodiments, the hydraulic system can further comprise a hydraulic fluid reservoir, the hydraulic fluid reservoir in communication with the first fluid path and the second fluid path.

Broadly stated, in some embodiments, the hydraulic fluid reservoir can comprise a stand-alone reservoir external to the cylinder.

Broadly stated, in some embodiments, the hydraulic fluid reservoir can be comprised of a forward chamber and a back chamber disposed in the cylinder, the forward and back chambers separated by the piston.

Broadly stated, in some embodiments, the hydraulic system can further comprise a fluid pump disposed in the first fluid path, wherein operation of the pump can move the hydraulic fluid into the cylinder to move the piston upwards therein, thereby moving the arm cuff to the elevated position.

Broadly stated, in some embodiments, the shoulder brace can be used by the person to hold the arm in the elevated position when the shoulder is injured or when the shoulder cannot support the arm in the elevated position.

### BRIEF DESCRIPTION OF THE DRAWINGS

FIG. 1 is a front elevation view depicting a person wearing a dynamic shoulder brace properly affixed to the body with arms at a resting position.

FIG. 2 is a rear elevation view depicting a person wearing the dynamic shoulder brace of FIG. 1.

FIG. 3 is a front elevation view depicting a person wearing the dynamic shoulder brace of FIG. 1 using a flat surface to support the arm while moving away to lock the brace into the elevated position.

FIG. 4 is a front elevation view depicting a person wearing the dynamic shoulder brace of FIG. 3 with the arm positioned in the elevated position showing full extension of the arm away from the body.

FIG. 5 is a front elevation view depicting a person wearing the dynamic shoulder brace of FIG. 4 with the arm positioned in the elevated position showing the externally rotated arm pointing in an upward position.

FIG. 6 is a front elevation view depicting a person wearing the dynamic shoulder brace of FIG. 4 with the arm positioned in the elevated position showing the arm pointing in a forward position.

FIG. 7 is a front elevation view depicting a person wearing the dynamic shoulder brace of FIG. 6 with the arm positioned in the elevated position showing the arm pointing in a forward position and also showing the actuation of the pull ring by the person's other hand.

FIG. 8 is an elevation view depicting one embodiment of a dynamic shoulder brace.

FIG. 9 is an isometric view depicting the dynamic shoulder brace of FIG. 8.

FIG. 10 is a front elevation cutaway view depicting one embodiment of a ratchet mechanism for use with the dynamic shoulder brace of FIG. 8.

FIG. 11 is a rear elevation view depicting the ratchet mechanism of FIG. 10 showing a spring and extended ratchet release lever.

FIG. 12A is a block diagram depicting an alternate embodiment of a mechanism for raising an arm in a dynamic shoulder brace, wherein a hydraulic mechanism is used in place of a ratchet mechanism.

FIG. 12B is a block diagram depicting a second embodiment of a hydraulic mechanism for a dynamic shoulder brace where the arm is being elevated.

FIG. 12C is a block diagram depicting the hydraulic mechanism of FIG. 12B where the arm is being lowered.

FIG. 13 is a block diagram depicting a wireless remote and brace controller for use with a dynamic shoulder brace.

FIG. 14 is a block diagram depicting a wired remote for use with a dynamic shoulder brace.

FIG. 15A is a rear elevation cutaway view depicting one embodiment of the ratchet mechanism of FIG. 10 comprising strain gauges and an actuator to release the ratchet mechanism, the ratchet mechanism shown in a fixed and non-released position.

FIG. 15B is a rear elevation cutaway view depicting the ratchet mechanism of FIG. 15A with the actuator releasing the ratchet mechanism to allow downward movement of the arm shaft support.

FIG. 16A is a rear elevation view depicting an embodiment of a dynamic shoulder brace comprising a lifting mechanism incorporating the hydraulic system of FIGS. 12B and 12C where the arm support shaft is in an elevated position.

FIG. 16B is a rear elevation view depicting the lifting mechanism of FIG. 16A where the arm support shaft is in a lowered position.

FIG. 17A is a rear elevation view depicting an alternate embodiment of lifting mechanism of FIG. 16A.

FIG. 17B is a rear elevation view depicting the lifting mechanism of FIG. 17A where the arm support shaft is in a lowered position.

FIG. 18 is a block diagram depicting one embodiment of a hydraulic fluid circuit for use with the lifting mechanism shown in FIGS. 16A, 16B, 17A and 17B.

### DETAILED DESCRIPTION OF EMBODIMENTS

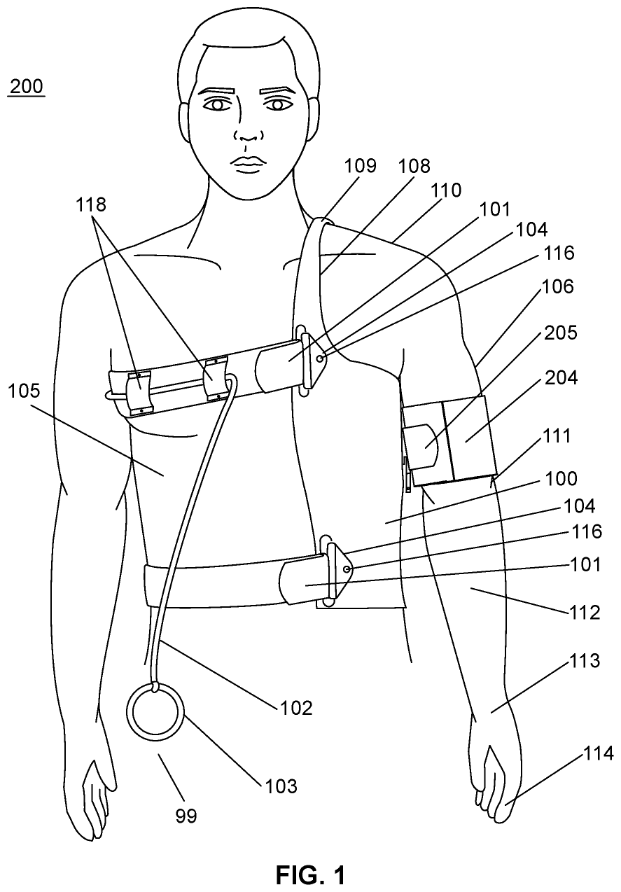

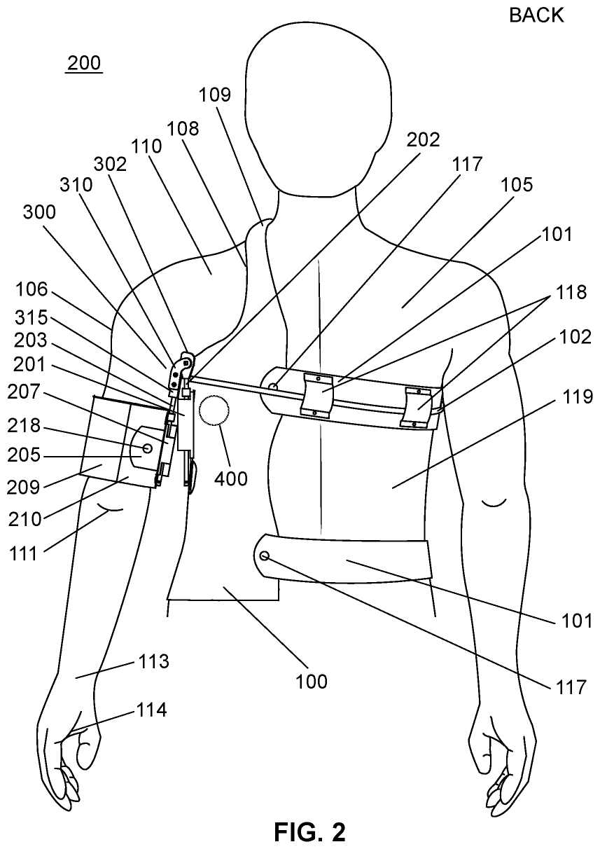

A dynamic shoulder brace (200) to provide temporary stable support to an elevated arm of a user while maintaining significant movement flexibility to the user is provided. Referring to FIGS. 1 and 2, in some embodiments, shoulder brace (200) can comprise of a chest piece configured to be attached to a patient's body; an arm cuff configured to be attached to the patient's upper arm; a lifting mechanism operatively coupled between the chest piece and the arm cuff, the lifting mechanism configured to permit upward vertical movement of the patient's arm pivoting upwardly about the patient's shoulder joint, the lifting mechanism further configured to hold the patient's arm in an elevated position; and a release mechanism configured to operate the lifting mechanism in a manner to allow the patient to lower their arm from the elevated position. The chest piece can be molded or custom-fitted to the patient's body or torso in a manner as well known to those skilled in the art.

In some embodiments, brace (200) can comprise chest piece (100) coupled to a lifting mechanism that can comprise of vertical support member (201) with integral vertical sliding shaft (202) coupled to arm support shaft (203) via ratchet mechanism (300) that can be loosely coupled to support shaft attachment slide (207), wherein attachment slide (207) can be rigidly connected to arm cuff (204).

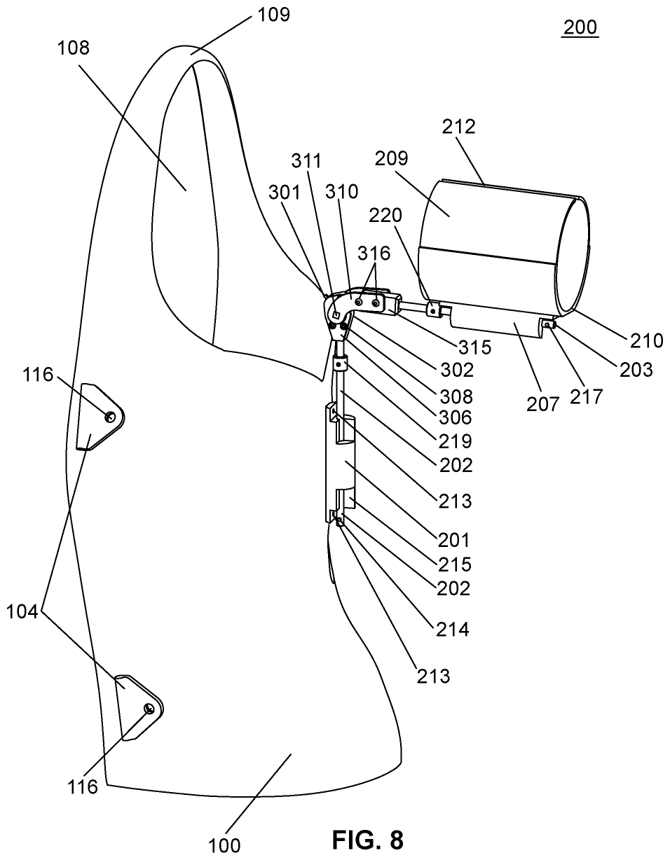

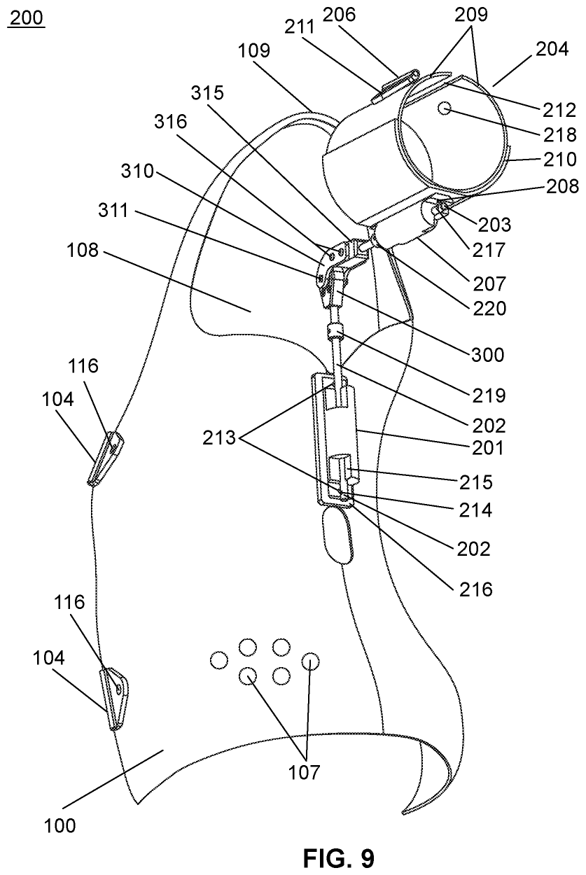

With reference to FIGS. 8 and 9, in some embodiments, dynamic shoulder brace (200) can comprise molded form fitting chest piece (100) outfitted with clasps (104) and tightening straps (101). In some embodiments, tightening straps (101) can comprise hook and loop fasteners, such as those made by Velcro® but can also be made of a natural or synthetic belt material as well known to those skilled in the art. The material can be breathable or comprise perforations to provide additional comfort to the user when wearing brace (200). In embodiments comprising hook and loop fasteners, clasp (104) can comprise a metallic or synthetic ring that hook and loop fastener strap (101) can loop through before fastening back on itself to tighten. For those embodiments with straps (101) made of a natural or synthetic belt material, clasp (104) can comprise of a variety of clasp (104) mechanisms and materials known to those skilled in the art that can include, but are not limited to, buckles, D-rings, side snap clips and other functionally equivalent fastening mechanisms that comprise tightening provisions. In some embodiments, clasps (104) can be attached to chest piece (100) using adhesives, welding (ultrasonic or otherwise) or strap clasp mounting hardware (116) that can comprise of snaps, screws and nuts, barbed inserts, rivets, bolts, or any other suitable hardware known to those skilled in the art. In some embodiments, straps (101) can be sufficiently wide so as to provide a greater surface area in which to distribute the tightening pressure and increase user comfort. Some embodiments can use many small straps to accomplish a similar result by having many small anchor points treaded together as one strap or lace like system. As shown in FIG. 2, straps (101) can be mounted to the other side of chest piece (100) opposite of clasp (104) using adhesives, welding (ultrasonic or otherwise, or strap mounting hardware (117) such as but not limited to snaps, rivets, or other suitable hardware, or any combination thereof known to those skilled in the art. In some embodiments, straps (101) can be mounted at the rear side (patient's back side) of chest piece (100), and clasps (104) can be mounted on the front side of chest piece (100) to provide ease of access to the user. In some embodiments, the number of straps (101) and clasps (104) can be equal. In some embodiments, chest piece (100) can comprise upper shoulder support (109) that can sit on user's shoulder (110) in addition to arm aperture (108) through which the user's arm (106) can be inserted through.

In some embodiments, brace (200) can comprise of release mechanism (99). In the illustrated embodiment, release mechanism (99) can comprise pull string (102) disposed either inside channel (118) incorporated into strap (101), or through a tube or conduit that can be added to strap (101). A tube or conduit can be made of a plurality of different materials but will generally be some form of a synthetic flexible tubing. Pull string (102) can be made of a variety of different materials such as but not limited to aircraft cable, insulated cable, or any kind of natural or synthetic cable or rope. In some embodiments, pull string (102) can be permanently or temporarily coupled to pull ring (103) that can be made of metallic or synthetic materials of any number of shapes, sizes, and colours depending on the desire of the orthotist and the user. Pull ring (103) can be attached to pull string (102) using a plurality of different techniques including, but not limited to, tying, clamping, welding, or any other method or technique that is known to those skilled in the art. Users with reduced strength and mobility in their hands may choose pull rings (103) that are larger and easier to grab, whereas younger users or users with more mobility may choose pull rings (103) that are smaller and more discrete. Straps (101) can be used to affix molded form fitting chest piece (100) to body (105). In other embodiments, other body affixation techniques, such as using a continuous fabric, can be used in substitution of molded chest piece (100). In other embodiments, other functionally equivalent body attachment devices as well known to those skilled in the art can be used in substitution of, or in combination with, molded chest piece (100).

In some embodiments, molded form fitting chest piece (100) can be made of high-density polypropylene or other suitable form-fitting thermoplastic and manufactured by an orthotist using practices, procedures, and materials well known to those skilled in the art. The size and shape of chest piece (100) inclusive of shoulder support (109) and arm aperture (108) will be customized for each patient's body (105), shoulder (110) size, and arm (106) diameter with strap (101) and clasp (104) positioned in accordance with the experience and knowledge of the orthotist based on providing sufficient support whilst being comfortable to wear. The orthotist may add padding, plugs, covers, and foam around chest piece (100) to increase comfort and to hide access to the various hardware components (116, 117, 213) used to affix backing plate (216) and vertical support member (201) and clasps (104) to chest piece (100), as shown in FIGS. 1, 2, 8 and 9. Chest piece (100) can provide one or more holes that can be used to pass through vertical support attachment hardware (213) to align and support vertical support member (201). Vertical support member (201) can be used to provide a connection to vertical sliding shaft (202) that can further comprise ratchet mechanism (300) affixed to the top of vertical sliding shaft (202). Vertical support attachment hardware (213) can consist of adhesives, welding (ultrasonic or otherwise), snaps, screws and nuts, bolts and barbed insert type bushings, rivets, or any other suitable hardware known to those skilled in the art. Chest piece (100) can also comprise a number of small holes (107) disposed therethrough to provide ventilation and increase the comfort for the user. The user will also generally wear chest piece (100) on top of a shirt for comfort. Both vertical sliding shaft (202) and arm support shaft (203) can comprise additional collars (219, 220) that can limit total movement of brace (200) towards ratchet mechanism (300) and, depending on the material in which vertical support collar (219) and arm support shaft collar (220) are comprised of, these can also provide a softer bumper for vertical support member (201) and support shaft attachment slide (207) to impact against ratchet mechanism (300). Vertical support collar (219) and arm support shaft collar (220) can be sliding or permanently attached using various different affixation techniques as known to those skilled in the art. Vertical support collar (219) and arm support shaft collar (220) can be comprised of one or more of a number of metallic or non-metallic, synthetic or natural materials including, but not limited to, PTFE, acetal, anodized aluminum and stainless steel in addition to a variety of other materials known to those skilled in the art.

In some embodiments, user's arm (106) can be coupled to arm support shaft (203) through arm cuff (204) and support shaft attachment slide (207). In some embodiments, arm cuff (204) can comprise split opening (212) through which user's arm (106) can easily slide into cuff (204). Arm cuff (204) can comprise cuff strap (205) and cuff strap clasp (206) that can be used to tighten cuff (204) around user's arm (106). In some embodiments, cuff strap (205) can comprise of hook and loop fastener material such as those made by Velcro® but can also comprise a natural or synthetic belt material, wherein the material can be breathable, or contain perforations to provide additional comfort to the user when wearing cuff (204). In embodiments comprising hook and loop fastener material, cuff strap clasp (206) can comprise a metallic or synthetic ring that hook and loop fastener cuff strap (205) can loop through before folding back on itself to tighten. For those embodiments with cuff straps (205) made of a natural or synthetic belt material, cuff clasp (206) can comprise of a variety of cuff strap clasp (206) mechanisms and materials known to those skilled in the art that can include, but are not limited to, buckles, D-rings, and side snap clips with incorporated tightening provisions. In some embodiments, cuff straps (205) can be sufficiently wide so as to provide a greater surface area in which to distribute the tightening pressure and increase user comfort. In some embodiments, cuff (204) can be made from a flexible fabric or other structure suitable for rigidly attaching arm (106) to arm support shaft attachment slide (207).

In some embodiments, cuff strap clasp (206) can be mounted to one side of split opening (212) in arm cuff (204) using adhesives, welding (ultrasonic or otherwise), or cuff strap clasp mounting hardware (211) which can consist of snaps, screws and nuts, bolts and barbed inserts, rivets, or any other suitable hardware known to those skilled in the art. Cuff strap (205) can be mounted to the other side of split opening (212) in cuff (204) using adhesives, welding (ultrasonic or otherwise), or cuff strap mounting hardware (218) that can comprise one or more of snaps, screws and nuts, bolts, barbed inserts, rivets and other suitable and functionally equivalent hardware as known to those skilled in the art. Which side of split opening (212) in cuff (204) gets cuff strap (205) and cuff strap clasp (206) can be determined by the orthotist depending on which shoulder is being braced and the handedness of the user.

In some embodiments, arm cuff (204) can be made of a single material that incorporates arm support shaft attachment slide (207) as a single monolithic piece. However, in some embodiments, this can be difficult to produce as arm cuff (204) material needs to be flexible in order to bend around user's arm (106) whilst support shaft attachment slide (207) needs to be rigid and low friction in order to support the weight of arm (106) and to properly couple arm (106) to arm support shaft (203). In other embodiments, cuff (204) and arm support shaft attachment slide (207) can be separate pieces with cuff (204) comprise flexible cuff (209), which is in contact with user's arm (106), can be made of a flexible form-fitting thermoplastic material such as thin low density polypropylene that can be permanently affixed via adhesive or any other suitable bonding material known to those skilled in the art to rigid cuff support (210) material disposed on the bottom side to provide additional rigidity and support strength to the portion that is connected to arm support shaft attachment slide (207) via arm slide hardware (208). In some embodiments, rigid cuff support (210) can comprise any metallic or non-metallic, natural or synthetic material that is suitable for the application and, in some embodiments, can be made of a thermoplastic such as high-density polypropylene of a different density and rigidity than flexible cuff (209). Arm slide hardware (208) can consist of snaps, screws and nuts, bolts and barbed insert type bushings, rivets, or any other suitable hardware known to those skilled in the art. In some embodiments, support shaft attachment slide (207) can be comprised of any number of metallic, non-metallic, natural or synthetic materials, however, because its main function is to provide a low friction housing to arm support shaft (203), in some embodiments, support shaft attachment slide (207) must not be too heavy nor should it be absorbent of perspiration from the user. In some embodiments, plastic materials such as acetal, polytetrafluoroethylene, or polyether ether ketone (PEEK) can be used for support shaft attachment slide (207).

In some embodiments, arm support shaft end stop (217) can be disposed at the end of arm support shaft (203) that can comprise of one or more of the following attachments means: a pressed in pin in a hole disposed through the shaft, a bolt threaded through the shaft which may or may not also have a thread locker applied; a collar dog with set screw; a clamp; or any other similar mechanism known to those skilled in the art capable of preventing arm support shaft attachment slide (207) from becoming decoupled from arm support shaft (203).

In some embodiments, arm cuff (204), inclusive of flexible cuff (209) and rigid cuff support (210), can be fabricated for each individual user by an orthotist based on the user's individual arm dimensions; however, in some embodiments, it is possible to mass produce cuff (204) in advance in a variety of sizes that can, if necessary, be adjusted to a final shape by the orthotist during fabrication and assembly of the dynamic shoulder brace (200). The orthotist may add padding, plugs, covers, and foam around cuff (204) to increase comfort and to hide access to various hardware components (208, 211, 218) used to affix support shaft attachment slide (207) and clasps (206) to cuff (204).

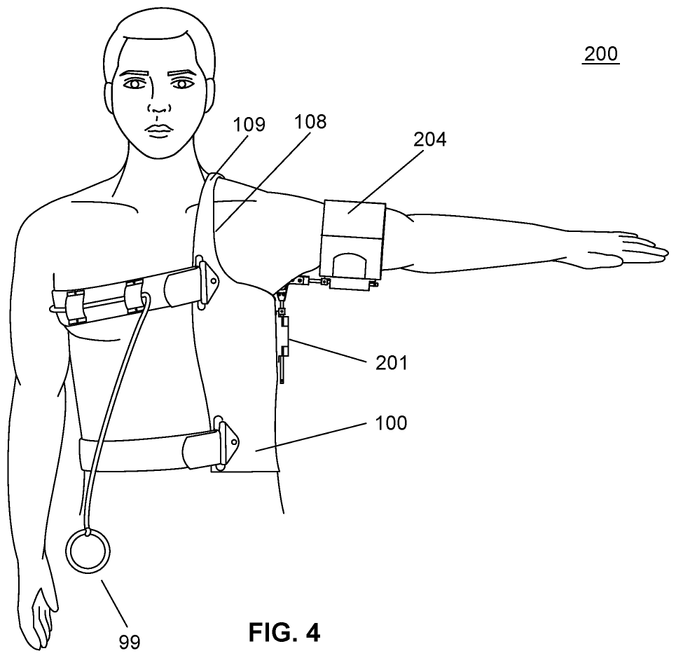

In some embodiments, pull string (102) can be coupled on one end to pull ring (103), wherein the other end can be coupled to ratchet release lever (301). In some embodiments, ratchet mechanism (300) can be designed such that it can ratchet and lock arm support shaft (203) preventing rotation in one direction but when ratchet release lever (301) is engaged by the user, arm support shaft (203) and user's arm (106) can be allowed to freely fall back to the normal resting position as shown in FIG. 1 from the elevated position shown in FIGS. 4, 5, 6 and 7.

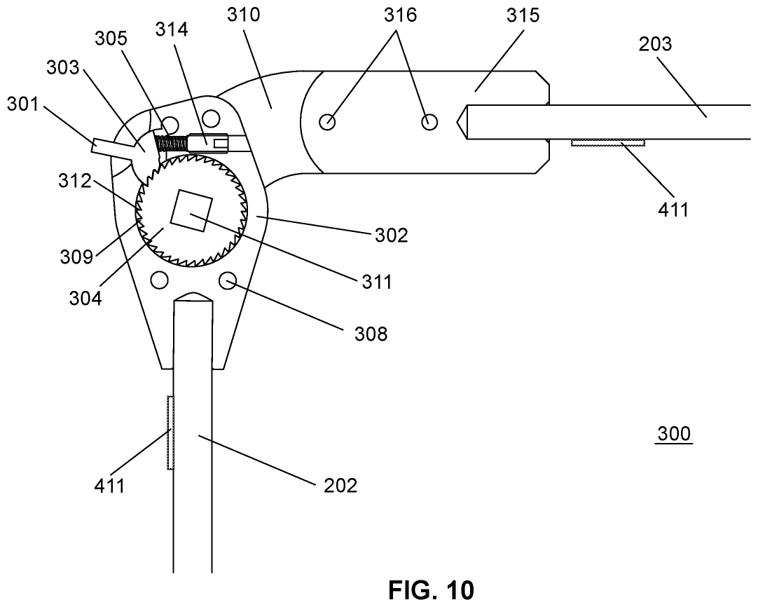

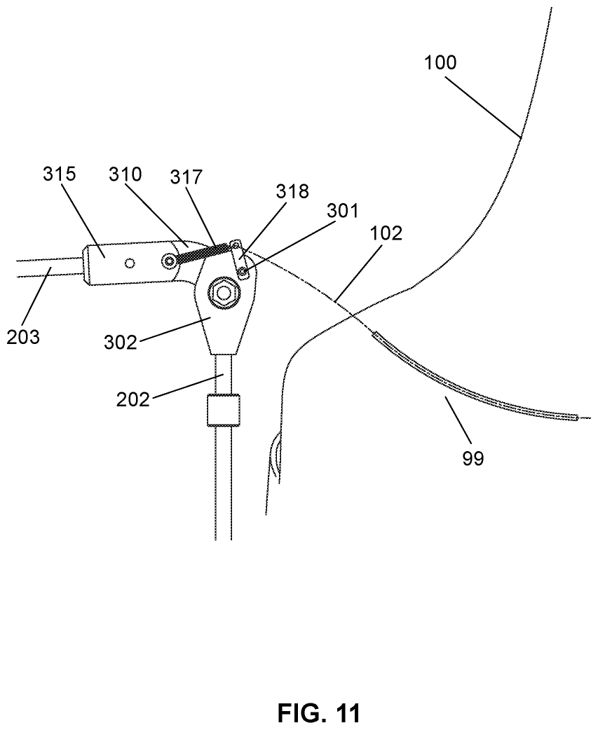

In some embodiments, as shown in FIG. 10, ratchet mechanism (300) can comprise of ratchet wheel (304) rigidly mounted to ratchet arm(s) (310) by means of square shaft (311). In some embodiments, ratchet arms (310) can connect to arm support shaft (203) or vertical sliding shaft (202), depending on orientation of ratchet mechanism (300). In other embodiments, square shaft (311) can be splined or keyed, or employ other mechanical means of fixing the internal components as known to those skilled in the art. Ratchet wheel (304) can be held within ratchet housing (302) where top lands (312) of ratchet wheel teeth (309) can provide the bearing surface for the radial forces generated by the weight of arm (106) in ratchet mechanism (300). In some embodiments, ratchet housing cover plate (306) can enclose ratchet wheel (304) and pawl (303) by bolting to ratchet housing (302) by means of cover fasteners (308). In other embodiments, bearings can be incorporated into the assembly to relieve the wear on ratchet wheel top lands (312). Ratchet wheel (304) can be allowed to rotate in one direction, and arrested in the opposite direction, by means of toothed pawl (303), whose teeth mesh into ratchet wheel (304). In the embodiment shown in FIG. 10, pawl (303) can be held in engagement with ratchet wheel (304) by the force applied by pawl spring (305), which can be held in place by set screw (314) within a bore of ratchet housing (302). In some embodiments, tooth (309) geometry can be such that engagement can pull pawl (303) and ratchet wheel (304) together while under load. Ratchet housing (302) can be configured to provide access for disengaging pawl (303) through mechanical means using release mechanism (99). In some embodiments, when the user engages pull ring (103), the force can be transmitted though pull string (102) to release lever (301). In some embodiments, such as shown in FIG. 11, internal pawl spring (305) can be replaced or supplemented with external release lever spring (317) attached on one end to ratchet arm (310), connector block (315), or on ratchet housing (302), and the other end can be connected to release lever extension arm (318) and pull string (102), with the opposite end of release lever extension arm (318) connected to release lever (301) to provide additional holding pressure and increase force required to disengage pawl (303). In some embodiments, release lever (301) can act on pawl (303) thereby releasing ratchet wheel (304) and, thus, allowing user's arm (106) to fall freely. When pawl (303) is disengaged through activation of release lever (301), ratchet wheel (304) can rotate freely in either direction. The weight from arm (106) coupled to arm cuff (204), as coupled to connection block (315) and upper ratchet arms (310), can force the mechanism to fall to its lowest energy state or lowered state. When pawl (303) is allowed to re-engage ratchet wheel (304), weight of arm (106) can be supported against rotation once again. In some embodiments, the assembly can be constructed completely from one or more of a combination of stainless steel, steel, brass, bronze, aluminum and plastics. In some embodiments, the base of ratchet housing (302) can comprise a hole for affixing either vertical sliding shaft (202) or arm support shaft (203) depending on orientation. In the embodiment shown in FIG. 10, vertical sliding shaft (202) can be attached to ratchet housing (302), and arm support shaft (203) can be attached to ratchet arms (310) through connection block (315) using fasteners (316). Other embodiments can comprise connection block (315) and ratchet arms (310) as one homogenous piece with arm support shaft (203) attached using techniques such as arc welding, friction welding, shrink fit, threaded connection, brazing or a wide variety of other methods known to those skilled in the art. In some embodiments, vertical sliding shaft (202) can also be attached to ratchet housing (302) using techniques comprising one or more of arc welding, friction welding, shrink fit, threaded connection, brazing and other functionally equivalent methods known to those skilled in the art. In some embodiments, ratchet arms (310) can comprise a single arm rather than two as is shown in FIG. 11.

In some embodiments, the single rotation direction may be accomplished by use of a sprag bearing or “one-way-bearing.” A release mechanism can be constructed to allow the bearing to rotate in its housing or, alternatively, release the sprags. In some embodiments, one or both of a brake mechanism and a clutch can be incorporated therein.

In some embodiments, ratchet wheel (304) can be, alternatively, incorporated into ratchet housing (302) by machining or broaching an internal tooth profile. Pawl (303) can then be incorporated into ratchet arms (310) preventing rotation in one direction until release lever (301) is engaged.

In some embodiments, pawl (303) can be disposed on a rocker or pin rather than rotating in its housing. In addition, in some embodiments, pawl (303) can comprise a series of compliant mechanisms formed through electrical discharge machining into housing (302).

In some embodiments, pawl (303) can comprise a different means of disengaging apart from a release lever. In some embodiments, there can be a cable rigidly fixed to pawl (303) that can run through pawl spring (305) and a hollow set screw. Pulling directly on pawl (303) to overcome pawl spring (305) can release ratchet wheel (304) and associated mechanism and arm support shaft (203).

In some embodiments, it can be desirable to increase the resolution of the increments of the ratcheting mechanism. This can be accomplished by using multiple pawls. In these embodiments, only one pawl can be engaged while the other pawls skip over to the next set of teeth disposed on a ratchet wheel. Alternatively, a gear ratio can be employed to provide multiple rotations of a ratchet wheel while going through the same travel of typical arm (106) movement.

In some embodiments, ratchet wheel (304) can only be partially cut for teeth (309) since the engagement rotation can only be around 120 degrees for typical arm (106) movement. The other 240 degrees of rotation can be left uncut and provide a better bearing surface for forces to transmit into ratchet housing (302).

In some embodiments, multiple parts can be 3D printed into the body of the ratchet housing. This can prove beneficial in reducing the overall number of parts. Pawl springs (305) and pawl(s) (303) can be directly printed into housing (302) while still providing good mechanical properties in a compliant manner.

In some embodiments, ratchet wheel (304) can, instead, comprise a pair of discs with ratcheting teeth cut into their mating faces. When the user decides to let down arm (106), the mechanism can disengage the two mating discs from each other and, thereby, allowing arm (106) to descend.

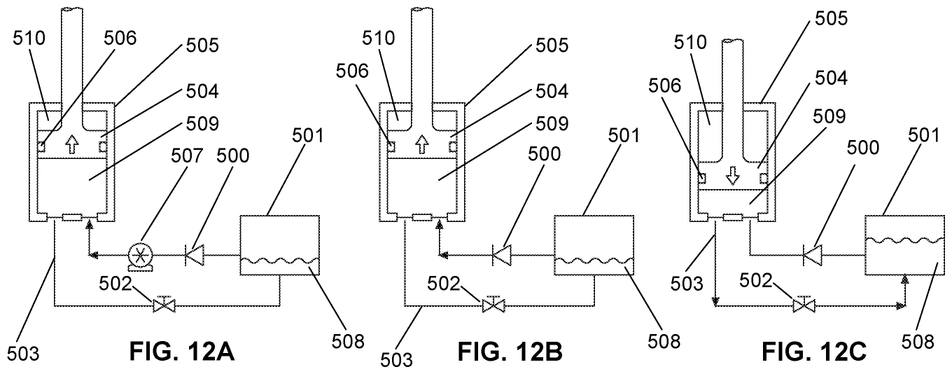

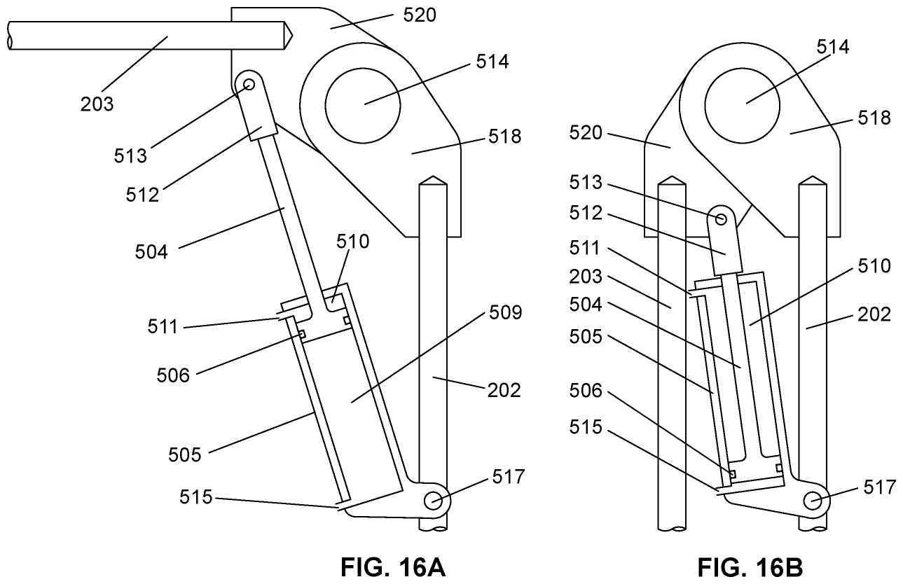

In some embodiments, as shown in FIGS. 12A, 12B and 12C, the mechanism to arrest the downward motion of arm (106) can comprise a hydraulic system comprising of check valve (500), reservoir (501), release valve (502) and piping or hose (503) to connect components together. In some embodiments, as shown in FIG. 12B, the user, while manually lifting arm (106), can lift piston (504) with seal (506) coupled to arm support rod (203) within cylinder (505) thereby drawing hydraulic fluid (508) into forward chamber (509). Upon the user lowering arm (106), piston (504) can attempt to push fluid (508) out of forward chamber (509), however, fluid (508) would not be allowed to back flow due to check valve (500) and normally-closed release valve (502) held in the closed position. With no escape for fluid (508) from forward chamber (509), arm support rod (203) coupled to piston (504) can securely fix user's arm (106) in the desired position. When the user has completed the tasks at hand and wishes to return arm (106) to the relaxed position, the user can open release valve (502). As shown in FIG. 12B, opening release valve (502) can allow fluid (508) to flow from forward chamber (509) back into reservoir (501), pushed by the weight of user's arm (106) acting on piston (504). Some embodiments can include an adjustment valve to control the rate of decent at varying speeds, or simply include an orifice plate to provide a controlled lowering of the arm, located after or before release valve (502). The adjustment valve or orifice plate would act as restrictors to fluid (508) flowing back into reservoir (501) and control the speed at which arm (106) is lowered. In other embodiments, release valve (502) can have enough fine adjustment built in to accomplish this goal without need for additional components. Some embodiments can include pump (507) to lift arm (106) without need for user exertion or provide a mechanical advantage to the user. Pump (507) can be of various makes or styles such as a centrifugal or positive displacement pump, either manually or electrically actuated. For example, a manually actuated piston pump can be added to the system to provide some fine adjustment in arm height with use of the system shown in FIG. 12A. In some embodiments, there can be a double acting piston that can shift hydraulic fluid (508) between forward chamber (509) and back chamber (510). In this embodiment, hydraulic fluid (508) can flow from back chamber (510) through check valve (500) into forward chamber (509) as the user raises arm (106). Check valve (500) can prevent back flow of hydraulic fluid (508) locking the device and, therefore, can prevent arm (106) from lowering. When the user opens release valve (502), hydraulic fluid (508) can then have a path to flow from forward chamber (509) into back chamber (510). In some embodiments, having hydraulic fluid flow from front chamber (509) to back chamber (510) and vice versa can eliminate the requirement for a larger separate reservoir (501). In some embodiments, pressure sensors can be installed to determine force feedback values that can provide pertinent information for the user or the user's therapy team. In some embodiments, hose or piping (503) can be replaced by internal channels integrated into cylinder (505) during its manufacture, eliminating the requirement for hose or piping (503). In some embodiments, the hydraulic fluid reservoir described herein can comprise a stand-alone container, as designated as reservoir (501) as shown in FIGS. 12A, 12B and 12C. In other embodiments, as shown in FIGS. 16A, 16B, 17A, 17B and 18, the hydraulic fluid reservoir described herein can comprise a combination of forward chamber (509) and back chamber (510) as the mechanism for holding hydraulic fluid (508) for use by the mechanism for arresting the downward motion of arm (106) thereby negating the need for stand-alone reservoir (501).

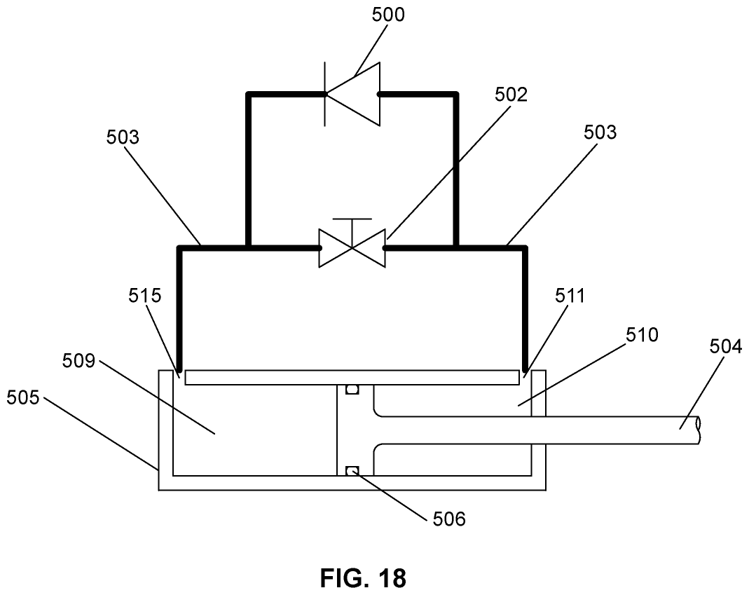

Presented in FIGS. 16A and 16B is an embodiment of a lifting mechanism for use with brace (200) comprising hydraulic piston (504) complete with rod end (512) and hydraulic seal (506), held within cylinder (505) complete with inlet (515) and outlet (511). The hydraulic system (as shown in FIGS. 12A, 12B and 12C) can be coupled to vertical support (202) and arm support (203) through a pinned or compliant connection. In the illustrated embodiments shown in FIGS. 16A and 16B, piston (504) can be connected to arm rod connection block (520) via rod end (512) and pin (513). Similarly, cylinder (505) can be connected to vertical support member (202), via pin (517) connection, which can be rigidly coupled to vertical connection block (518). As piston (504) is actuated upwards or downwards, pins (513, 517) and main pivot (514) can rotate about their axis. In the down position, as shown in FIG. 16B, the user would have their arm (106) in a relaxed or down position while in FIG. 16A, the user would have their arm (106) at a 90 degree position relative to their body (105). In reference to the hydraulic schematic in FIG. 18, in order for piston (504) and, therefore, user's arm (106) to be held in position in FIG. 16A, check valve (500) can be located on inlet (515) of cylinder (505). In some embodiments, as shown in FIG. 18, bypass or release valve (502) can be located parallel to check valve (500) to allow fluid (508) to flow back through inlet (515), through release valve (502), and into outlet (511) allowing piston (504) to lower.

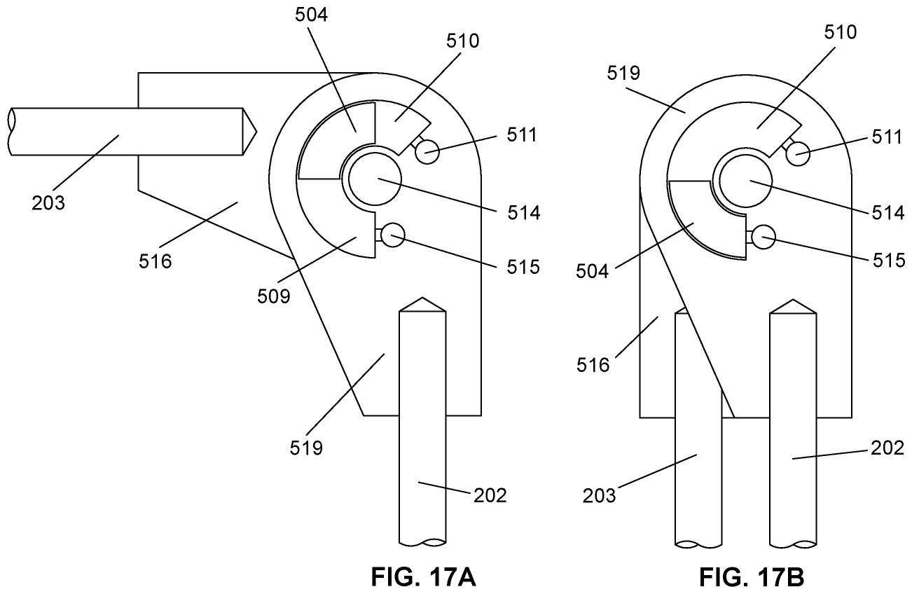

In some embodiments, as shown in FIGS. 17A and 17B, the hydraulic system can be integrated into main pivot (514) and arm support connection blocks (516) of the hydraulic locking mechanism (as shown in FIGS. 12A, 12B and 12C). FIG. 17A illustrates an elevated position for arm (106) whereas FIG. 17B illustrates a lowered position for arm (106). When the user begins to raise arm (106), check valve (500) coupled to inlet (515) can allow hydraulic fluid (508) to enter front chamber (509) in piston chamber vertical connection block (519). The user can place weight back on shoulder brace (200) at any time and check valve (500) can prevent back flow and arrest the downward movement of user's arm (106). When the user reaches working height as shown in FIGS. 17A and 18, check valve (500) can arrest backflow of hydraulic fluid (508) and can hold user's arm (106) in place. As shown in FIG. 18, bypass or release valve (502) can be located parallel to check valve (500) and can allow fluid (508) to flow back through inlet (515), through release valve (502), and into outlet (511), allowing user's arm (106) to fall back to a lowered position as is shown in FIG. 17B.

In some embodiments, brace (200) can comprise a plurality of covers and guards, telescoping and non telescoping, disposed over ratchet mechanism (300) and the vertical and arm support components that can prevent the user's clothing or skin from being caught in the various mechanisms of brace (200).

### Using the Dynamic Shoulder Brace

In some embodiments, to attach dynamic shoulder brace (200) to body (105), the user first ensures that straps (101) and clasps (104) are free and unlinked, and that cuff straps (205) are loose enough to fit users' arm (106). The user can then place their arm (106) through arm aperture (108) on chest piece (100) and, at the same time, into arm cuff (204). The user can then slide arm (106) down through arm cuff (204) while raising chest piece (100) closer to body (105) to the point where shoulder support (109) sits on user's shoulder (110) as shown in FIG. 1. The user can now put first strap (101) through first clasp (104) and pull tight to stabilize while remaining comfortable and if using a hook and loop fastener fold over to attach first strap (101) to itself or, if using some other kind of strap (101) material, latch first clasp (104) as required. The user can then repeat the above steps for all the other straps (101) and clasps (104) as required after which chest piece (100) should be comfortably attached to body (105). With chest piece (100) attached, arm cuff (204) can be attached to arm (106). This is completed by ensuring that arm (106) is in a comfortable position hanging straight down as shown in FIG. 1 with arm (106) located in arm cuff (204) such that a portion of the users' biceps and triceps are located inside arm cuff (204). The user can now place (if not already placed) first cuff strap (205) through first cuff strap clasp (206) and pull tight to stabilize while remaining comfortable and if using a hook and loop fastener fold over and attach first cuff strap (205) to itself, or if using some other kind of cuff strap (205) material, latch first clasp (104) as required. Should there be more than a single cuff strap (205), then tighten the remaining cuff straps (205) sequentially to securely fasten arm cuff (204) to arm (106). Dynamic shoulder brace (200) is now successfully attached to body (105) and arm (106) and is ready for use. The above instructions are for an individual putting dynamic shoulder brace (200) on themselves, some users because of mobility, flexibility, or dexterity issues may require the assistance of another individual to successfully attach dynamic shoulder brace (200) to body (105) and arm (106). The instructions for this are the same as above but the assistant will likely help with positioning and supporting arm (106) and chest piece (100) and in doing up straps (101, 205).

Dynamic shoulder brace (200) allows free movement at a table or lowered height in that arm (106) is unobstructed in external and internal rotation of arm (106), abduction and adduction of arm (106) at shoulder (110), full normal flexion and extension of forearm (112) relative to upper arm at elbow (111) and rotation of the forearm (112) and wrist (113). Because of the conditions that dynamic shoulder brace (200) is used to treat, free flexion and extension of arm (106) at shoulder (110) is not possible as the user has reduced capability in this area and cannot do these movements without support. The freedom of movement of external and internal rotation of arm (106) above is possible because vertical sliding shaft (202) is allowed to rotate freely inside of vertical support member (201). Vertical sliding shaft (202) can be made of any number of metallic, non-metallic, natural or synthetic materials; however, in some embodiments, vertical sliding shaft (202) can be comprised of stainless steel whilst also being able to be polished, cleaned and sterilized with minimal corrosive effect. In some embodiments, vertical sliding shaft (202) can be held captive in vertical support member (201) by attached ratchet housing (302) on the top end of the shaft, and removable vertical shaft end stop (214) on the bottom end of vertical sliding shaft (202). Vertical shaft end stop (214) can comprise of, or be a combination of, but are not limited to, a pressed in pin in a hole through the shaft, bolt threaded through the shaft which may or may not also have a thread locker applied, collar dog with set screw, a clamp and any other similar mechanism known to those skilled in the art capable of preventing vertical sliding shaft (202) from becoming decoupled from vertical support member (201). In some embodiments, vertical shaft end stop (214) can be designed to only come out one side of vertical sliding shaft (202) such that movement restriction slot (215) inside of vertical support member (201) can restrict movement of the shaft to allow only a permitted amount of external and internal rotation of arm (106) while in certain positions. In some embodiments, vertical support member (201) can be comprised of any number of metallic, non-metallic, natural or synthetic materials; however, because its main function is to provide a low friction housing to the vertical sliding shaft (202) and must not be too heavy or absorbent of perspiration from the user, plastic materials such as acetal, polytetrafluoroethylene, or polyether ether ketone (PEEK) are particularly well suited. In some embodiments, vertical support member (201) can be coupled directly to chest piece (100) using support hardware (213) or, depending on the shape of body (105), adapter plate (216) can be required to properly space and provide accommodation for a non-flat surface, or specific positional requirements to attach vertical support member (201) to chest piece (100).

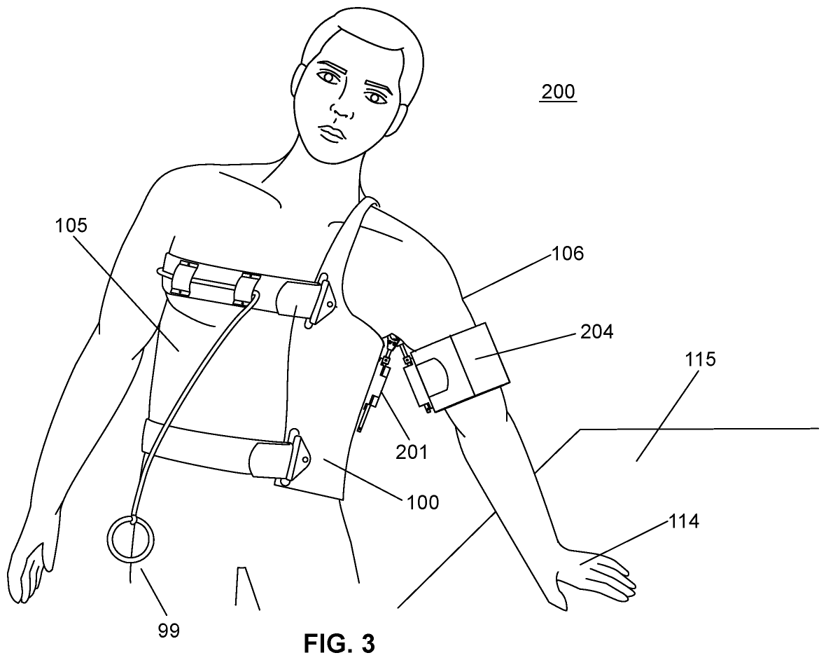

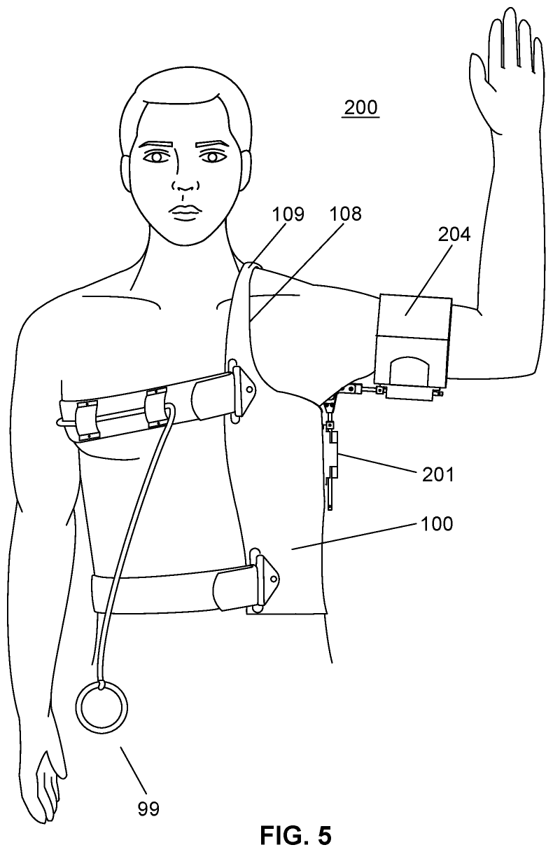

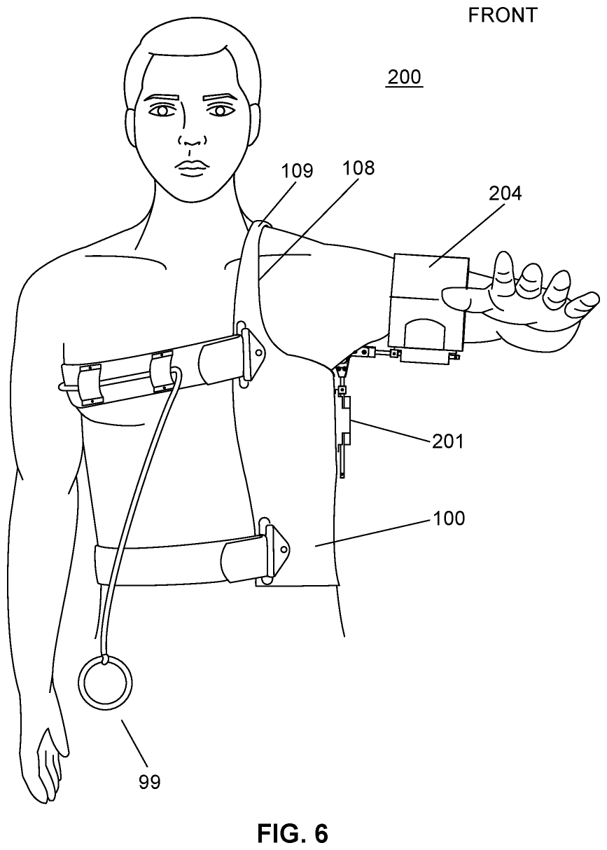

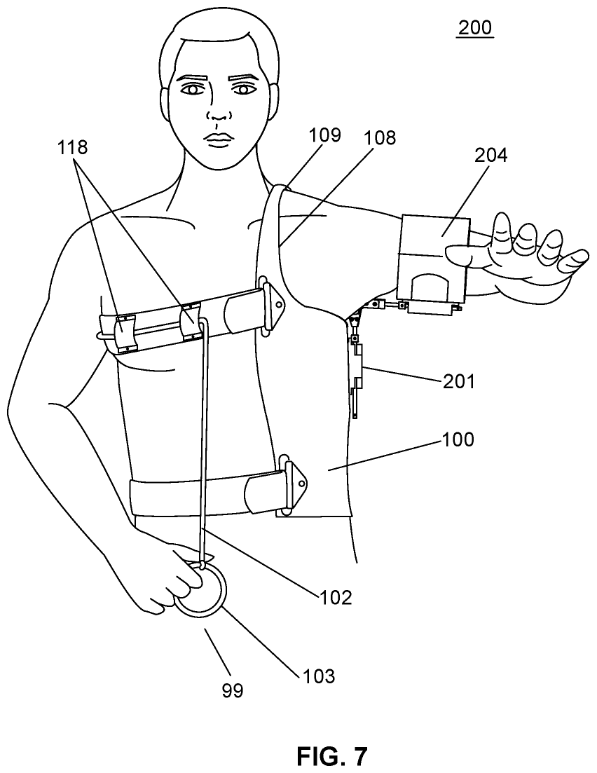

Users with reduced flexion and extension capabilities due to a shoulder injury will want to be able to lock their arm (106) into a elevated position to assist with any number of tasks such as, but not limited to, personal grooming such as shaving and hair brushing along with reaching and grabbing items out of elevated locations such as cupboards. As shown in FIG. 3, this can be achieved by securely placing hand (114) connected to arm (106) that is in cuff (204) on a flat surface or corner (115) and leaning away from arm (106) in such a way as to increase the angle between arm (106) and body (105). As the user increases this angle, ratchet mechanism (300) can advance. Arm support shaft (203), which can be rigidly coupled to ratchet wheel (304), will become locked in increasing fixed angles away from body (105) corresponding to tooth (309) increment locked in place by pawl (303). When the user has reached the desired angle of flexion between arm (106) and body (105), the user can stand up and arm (106) will be locked at that angle as shown in FIG. 4 by ratchet wheel (304) and pawl (303). In this position, the user has full internal and external rotation with full mobility at the elbow similar to the lowered position as shown in FIGS. 5 and 6. The angle between arm (106) and body (105) will generally never be more than 90 degrees from the lowered position. After the user has completed the task and wants to lower arm (106) to the lowered position as shown in FIG. 1, the user can activate release lever (301) by pulling with their non braced arm on pull ring (103) coupled to pull string (102) (as shown in FIG. 7) which can be coupled to release lever (301) and, in turn, pawl (303) can be disengaged from ratchet wheel (304) and ratchet wheel (304) can then be allowed to rotate in either direction and will no longer support user's arm (106). Release lever (301) built into pawl (303), can pull pawl (303) out of engagement with ratchet wheel (304) and the assembly can then be allowed to rotate in either the upward or downward directions. In some embodiments of ratchet mechanism (300), it may be required to slightly unload ratchet mechanism (300) by supporting and leaning arm (106) against something sturdy prior to pulling pull ring (103) as, in those embodiments, the tooth geometry can be such that in a loaded condition, ratchet wheel (304) and pawl (303) will be pulled into engagement with each other. This creates the condition where releasing ratchet mechanism (300) can require the unloading of arm support shaft (203) before release lever (301) on pawl (303) can be pulled with ease. In some embodiments, this angle can be flattened to prevent ratchet wheel (304) and pawl (303) from being pulled together. Release lever (301) can be pulled regardless of weight on the ratchet arms (310) with only a slight increase in resistance from the unloaded to loaded condition due to friction.

In some embodiments where the user may need to get larger heavier and fragile items regularly from a elevated position, it may be desirable for ratchet mechanism (300) to only lower by a single ratchet tooth distance with each activation of release lever (301) instead of having no support on the downward movement from a elevated position. Or in other embodiments, a hydraulic or pneumatic brake or release can be incorporated to provide a slow even decent as fluid is pushed through an orifice. Both of these embodiments can result in a more controlled, slower, descent of arm (106) from the elevated position.

Where the user has slightly more control and strength in the lower position, ratchet mechanism (300) can allow for free vertical movement for a specified number of degrees before ratchet mechanism (300) activates and can lock arm (106) in an elevated position. This can be implemented, in some embodiments, by pawl release lever (301) being released automatically in certain positions. This can be accomplished by means of a cam, where based on the position of the arm, can cause release lever (301) to remain engaged, releasing pawls (303) and allow both directions of rotation. This can also be accomplished electronically with actuator or solenoid (402) being used to release pawls (303). In some embodiments, an encoder can be attached to ratchet wheel (304) and receiver microcontroller (408) programmed to release or engage pawl (303) when required. This feature could also be used in therapy to challenge the user so they do not rely continuously on the device and as the user progressively gains strength the mechanism can progressively reduce the amount of support provided. Having this option would allow a user to be able to reach for things in a horizontal plane without moving their body if they have the strength and mobility to do so.

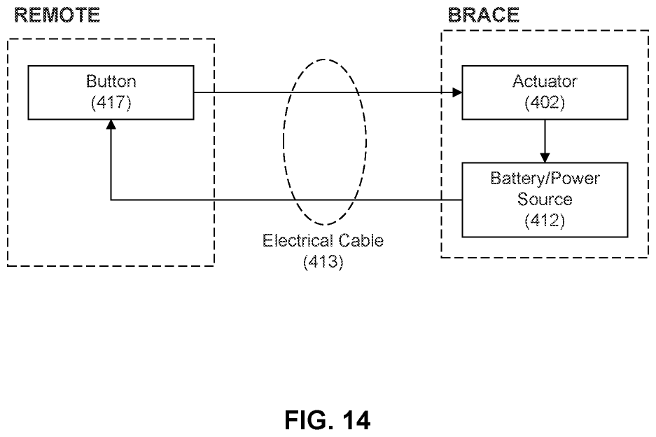

In some embodiments, as shown in FIG. 14, pull string (102) and pull ring (103) of release mechanism (99) can be replaced with button (417) operatively connected to actuator (402) via electrical cable (413), wherein actuator (402) can be mechanically coupled to release lever (301) and electrically connected to a plurality of different power sources such as, but not limited to, batteries (412). When the user presses button (417), a current carrying path between power source (412) and actuator (402) can be completed that can cause actuator (402) to actuate. In some embodiments, button (417) can be designed with a recessed plunger to prevent accidental activation. Actuator (402) can be composed of a plurality of different rotational or linear translation devices such as, but not limited to, linear motors and solenoids.

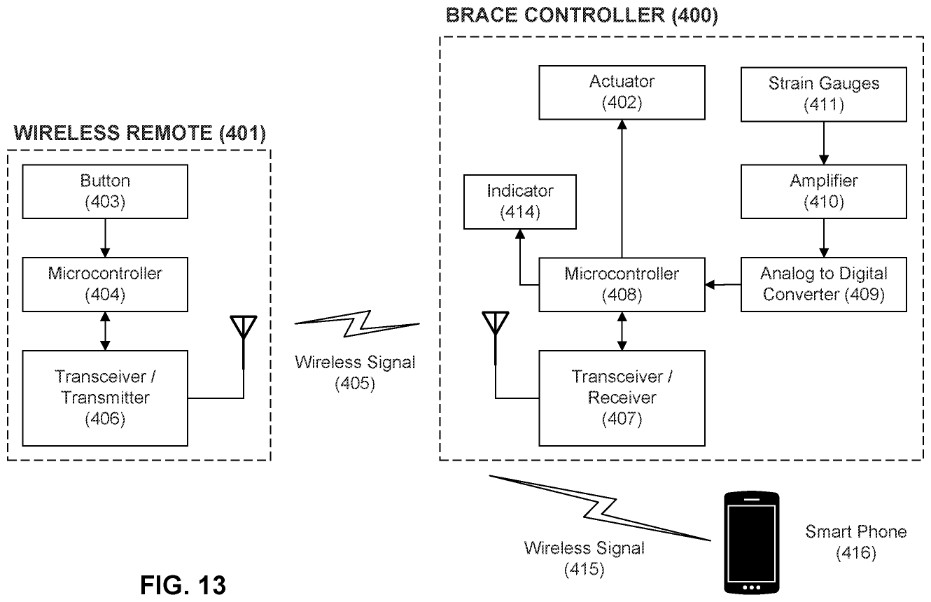

In some embodiments, as shown in FIG. 13, it can be desirable to not have pull string (102) to activate release lever (301) but, instead, have remote (401) wirelessly coupled to remote controller (400), wherein remote controller (400) can be coupled to actuator (402) that can be further connected to release lever (301). To activate release lever (301), the user can press button (403) on remote (401) that can then activate transmitter microcontroller (404) to transmit wireless signal (405) through transmitter antenna (406) that can then be received by receiver antenna (407) and then decoded appropriately by receiver microcontroller (408) and, if appropriate, can activate actuator (402) that can be coupled to release lever (301). In some embodiments, both remote (401) and controller (400) can be battery powered, and can further comprise visual or audible low battery indicators built therein. In some embodiments, remote (401) can be incorporated into functionality provided by smart phones (416) or smart watches and, in other embodiments, can be a separate piece of hardware. In some embodiments, wireless signal (405) can comprise one or more of a plurality of different communication hardware and protocols including, but not limited to, Bluetooth®, WiFi®, LoRa®, ASK, FSK, OOK, PSK and 5G technologies as well known to those skilled in the art. Both receiver (407) and transmitter (406) can be powered from integrated or removable, rechargeable or one time use, batteries.

In order to prevent accidental activation of remote (401), it can be desirable to have button (403) require a specific sequence such as, but not limited to, two or three sequential pushes each with at least 1s duration, or other period of time, to be depressed prior to initiating wireless signal (405) to receiver antenna (407). In some embodiments, transmitter remote (401) and receiver actuator (402) can be paired in such a way that only wireless signal (405) from approved remote (401) can be accepted as valid by receiver actuator (402) to prevent multiple remote equipped dynamic shoulder braces (200) from interfering with each other. This pairing can be done either at the protocol level such as, but not limited to, Bluetooth® pairing, or at an application level where the data can be encoded or encrypted prior to transmission with only the paired device having the key to decoding or decrypting the data indicating the desire to actuate actuator (402).

In other embodiments, the raising mechanism, instead of relying on the mobility of the user, can be driven by an electric actuator such as, but not limited to, a motor connected through a gear box operatively coupled to ratchet wheel (304) that can be powered by batteries and operated by remote (401) wherein arm (106) can be lifted by the motor turning ratchet wheel (304) such that ratchet mechanism (300) can still lock in place and prevent arm (106) from lowering until desired by the user. This embodiment can also include various inclinometers and other devices to ensure that the ratchet wheel (304) is not driven over the maximum angle which can be set for each patient.

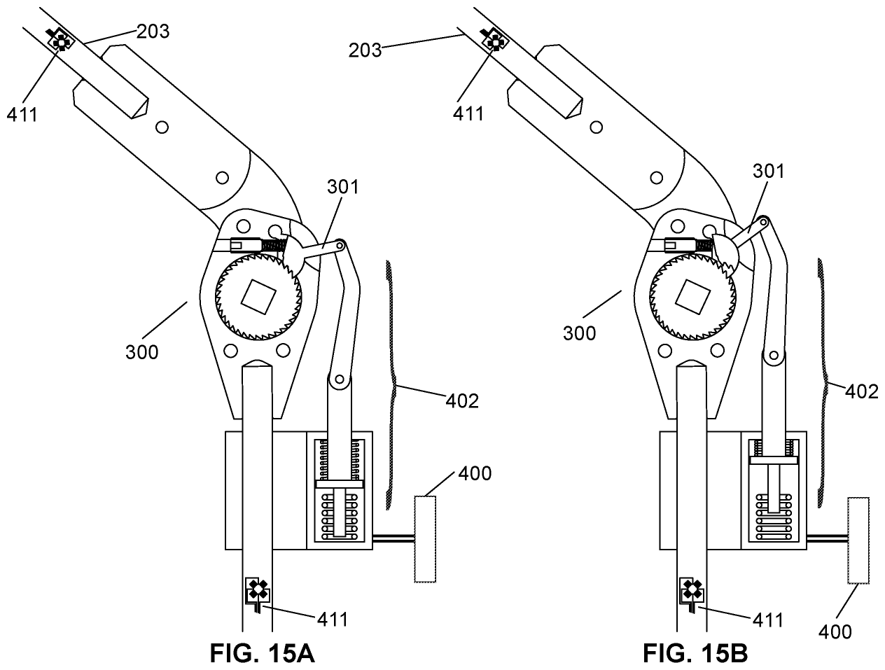

In some embodiments, dynamic shoulder braces (200) can be designed for a maximum load in which the brace (200) can be used before it receives permanent damage or an undue risk of injury to the user is created, this load includes both arm (106) being supported as well as the contents of the user's hands. Some embodiments of dynamic shoulder brace (200) can comprise strain gauges (411), as shown in FIGS. 10 and 15, or other suitable deflection or force measurement mechanisms configured to determine the current load the user is exerting on the brace mechanisms. Strain gauges (411) can be placed in a plurality of locations, but likely would be best located on arm support shaft (203) or on vertical sliding shaft (202). In some embodiments, the strain gauge signals can be first amplified using an instrumentation amplifier (410) that can provide signals suitable for input into an analog to digital converter (409) that can then be inputted into microcontroller (408) for processing and/or analysis. During manufacture of brace (200), strain gauges (411) can be calibrated in such a way as to allow microcontroller (408) to calculate the mass currently being supported. This mass can then be compared with a maximum and, if above the maximum, microcontroller (408) can turn on indicator (414) which can provide the user with feedback so they can lower the mass prior to damage to the brace mechanisms occurring. Devices that can be implemented as indicators (414) can include one or more of audible indicators such as speakers or piezo electric elements, vibratory indicators such as miniature motors with offset loads, and visual indicators in the form of a flash or light emitting diode (LED). In embodiments comprising a wireless lowering mechanism, microcontroller (408) can be configured to implement this mass monitoring system as described above. In some embodiments, wireless signal (415) can be sent to a user's smart phone (416) or other personal device to let them know the mass of the items they are holding and how close to the maximum load they are at.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of the embodiments described herein.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc. can be passed, forwarded, or transmitted via any suitable means including memory sharing, message passing, token passing, network transmission, etc.

The actual software code or specialized control hardware used to implement these systems and methods is not limiting of the embodiments described herein. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

Although a few embodiments have been shown and described, it will be appreciated by those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and described or portions thereof, it being recognized that the invention is defined and limited only by the claims that follow.

We claim:

1. A shoulder brace for a person having a torso, a shoulder and an arm attached to the shoulder, the shoulder brace comprising:
a) a chest piece configured for fitment and attachment to the torso;
b) an arm cuff configured for fitment and attachment to the arm;
c) an arm lifting mechanism operatively coupling the arm cuff to the chest piece, the arm lifting mechanism configured for upward movement of the arm cuff from a lowered position to a elevated position and holding the arm cuff in the elevated position thereby maintaining the arm at the elevated position; and
d) a release mechanism configured for acting on the arm lifting mechanism to enable downward movement of the arm cuff from the elevated position to enable the lowering of the arm.

2. The shoulder brace as set forth in claim 1, wherein the chest piece further comprises a shoulder support that supports the chest piece from the shoulder.

3. The shoulder brace as set forth in claim 1, wherein the chest piece comprises one or more tightening straps configured for releasable attachment of the chest piece to the torso.

4. The shoulder brace as set forth in claim 1, wherein the arm cuff comprises a cuff strap configured for releasable attachment of the arm cuff to the arm.

5. The shoulder brace as set forth in claim 1, wherein the arm lifting mechanism comprises:
a) a vertical support member operatively coupled to the chest piece;
b) a vertical sliding shaft slidably disposed within the vertical support member;
c) a shaft attachment slidably operatively coupled to the arm cuff;
d) an arm support shaft slidably disposed within the shaft attachment slide; and
e) a ratchet mechanism operatively coupling the vertical sliding shaft to the arm support shaft, the ratchet mechanism configured to enable upward rotational movement of the arm support shaft relative to the vertical sliding shaft when the arm cuff is moved from the lowered position to the elevated position.

6. The shoulder brace as set forth in claim 5, wherein the ratchet mechanism comprises:
a) a ratchet wheel operatively coupled to one of the vertical sliding shaft and the arm support shaft; and
b) a ratchet housing operatively coupled to the other of the vertical sliding shaft and the arm support shaft, the ratchet housing comprising a ratchet pawl, the ratchet pawl comprising a biasing element configured to urge the ratchet pawl to engage the ratchet wheel.

7. The shoulder brace as set forth in claim 6, wherein the release mechanism comprises:
a) a lever operatively coupled to the ratchet pawl; and
b) a pull string operatively coupled to the lever, the pull string configured to rotate the lever, wherein pulling the pull string disengages the ratchet pawl from the ratchet wheel enabling downward rotational movement of the arm support shaft relative to the vertical sliding shaft, and thereby enabling the arm cuff to move downward from the elevated position.

8. The shoulder brace as set forth in claim 7, wherein the pull string is disposed on the chest piece.

9. The shoulder brace as set forth in claim 6, wherein the release mechanism comprises:
a) a lever operatively coupled to the ratchet pawl; and
b) an actuator operatively coupled to the lever, the actuator configured to rotate the lever, wherein activation of the actuator disengages the ratchet pawl from the ratchet wheel enabling downward rotational movement of the arm support shaft relative to the vertical sliding shaft, and thereby enabling the arm cuff to move downward from the elevated position.

10. The shoulder brace as set forth in claim 9, wherein the actuator comprises an electrically-controlled solenoid.

11. The shoulder brace as set forth in claim 10, further comprising a battery and a wired remote button operatively coupled to the solenoid wherein depressing the wired remote button activates the actuator to release the ratchet pawl from the ratchet wheel.

12. The shoulder brace as set forth in claim 10, further comprising a wirelessly-controlled brace controller disposed thereon, the brace controller operatively coupled to the solenoid of the actuator, the brace controller configured to activate the actuator upon receiving a wireless signal.

13. The shoulder brace as set forth in claim 12, further comprising one or both of a wireless remote and a smart phone, each of the wireless remote and the smart phone configured to transmit the wireless signal to the brace controller.

14. The shoulder brace as set forth in claim 1, wherein the arm lifting mechanism comprises:
a) a vertical support member operatively coupled to the chest piece;
b) a vertical sliding shaft slidably disposed within the vertical support member;
c) a shaft attachment slidably operatively coupled to the arm cuff;
d) an arm support shaft slidably disposed within the shaft attachment slide, the arm support shaft rotatably coupled to the vertical sliding shaft; and
e) a hydraulic system operatively coupling the vertical sliding shaft to the arm support shaft, the hydraulic system configured to enable upward rotational movement of the arm support shaft relative to the vertical sliding shaft thereby enabling movement of the arm cuff from the lowered position to the elevated position.

15. The shoulder brace as set forth in claim 14, wherein the hydraulic system comprises:
a) a piston slidably disposed in a cylinder, the piston operatively coupled to the arm cuff;
b) a first fluid comprising a check valve configured to permit flow of the hydraulic fluid therethrough to the cylinder when the piston is drawn upward in the cylinder via the raising of the arm cuff to the elevated position, the check valve further configured to prevent the hydraulic fluid from flowing back therethrough from the cylinder; and
c) a second fluid path comprising a release valve configured to permit flow of the hydraulic fluid from the cylinder when the release valve is opened, wherein the piston moves downward in the cylinder thereby enabling the arm cuff to lower from the elevated position.

16. The shoulder brace as set forth in claim 15, wherein the hydraulic system further comprises a hydraulic fluid reservoir, the hydraulic fluid reservoir in communication with the first fluid path and the second fluid path.

17. The shoulder brace as set forth in claim 16, wherein the hydraulic fluid reservoir comprises a stand-alone reservoir external to the cylinder.

18. The shoulder brace as set forth in claim 16, wherein the hydraulic fluid reservoir is comprised of a forward chamber and a back chamber disposed in the cylinder, the forward and back chambers separated by the piston.

19. The shoulder brace as set forth in claim 15, wherein the hydraulic system further comprises a fluid pump disposed in the first fluid path, wherein operation of the pump moves the hydraulic fluid into the cylinder to move the piston upwards therein, thereby moving the arm cuff to the elevated position.

20. Use of the shoulder brace of claim 1 by the person to hold the arm in the elevated position when the shoulder is injured or when the shoulder cannot support the arm in the elevated position.
